(19) United States
(12) Patent Application Publication
(10) Pub. No.: US 2014/0018794 A1
(43) Pub. Date: Jan. 16, 2014

(54) OFF-WALL ELECTRODE DEVICES AND METHODS FOR NERVE MODULATION

(71) Applicant: BOSTON SCIENTIFIC SCIMED, INC., MAPLE GROVE, MN (US)

(72) Inventors: JAMES M. ANDERSON, FRIDLEY, MN (US); DEREK C. SUTERMEISTER, HAM LAKE, MN (US); HUISUN WANG, MAPLE GROVE, MN (US)

(21) Appl. No.: 13/941,242

(22) Filed: Jul. 12, 2013

Related U.S. Application Data
(60) Provisional application No. 61/671,536, filed on Jul. 13, 2012.

(51) Int. Cl. A61B 18/14

(52) U.S. Cl. CPC A61B 18/1492
USPC 606/41

(57) ABSTRACT

Systems for nerve modulation are disclosed. An example system for nerve modulation may include a catheter shaft having a proximal end, a distal end and lumen extending therebetween. An inflatable member may be fluidly connected to the lumen of the catheter shaft proximate the distal end of the catheter shaft. The inflatable member may have an outer wall defining a groove in an outer surface of the inflatable member. An electrode may be disposed in or under the groove.

### CROSS-REFERENCE TO RELATED APPLICATIONS

This application claims priority under 35 U.S.C. §119 to U.S. Provisional Application Ser. No. 61/671,536, filed Jul. 13, 2012, the entirety of which is incorporated herein by reference.

### TECHNICAL FIELD

The present disclosure relates to methods and apparatuses for modulating nerves through the walls of blood vessels. Such modulation may include ablation of nerve tissue or other modulation technique.

### BACKGROUND

Certain treatments require temporary or permanent interruption or modification of select nerve functions. One example treatment is renal nerve ablation, which is sometimes used to treat conditions related to congestive heart failure. The kidneys produce a sympathetic response to congestive heart failure, which among other effects, increases the undesired retention of water and/or sodium. Ablating some nerves running to the kidneys may reduce or eliminate this sympathetic function, providing a corresponding reduction in the associated undesired symptoms. For example, a renal nerve ablation procedure is often used to lower the blood pressure of hypertensive patients.

Many nerves (and nervous tissue such as brain tissue), including renal nerves, run along the walls of or in close proximity to blood vessels and these nerves can be accessed intravascularly through the blood vessel walls. In some instances, it may be desirable to ablate or otherwise modulate perivascular renal nerves using a radio frequency (RF) electrode. Such treatment, however, may result in thermal injury to the vessel at the electrode and other undesirable side effects such as, but not limited to, blood damage, clotting, and/or protein fouling of the electrode. To prevent such undesirable side effects, some techniques attempt to increase the distance between the vessel walls and the electrode. In these systems, however, the electrode may inadvertently contact the vessel walls.

Therefore, there remains room for improvement and/or alternatives in providing systems and methods for intravascular nerve modulation.

### SUMMARY

The disclosure is directed to several alternative designs and methods of using medical device structures and assemblies.

Accordingly, some embodiments pertain to a system for nerve modulation including a device having an inflatable member at the distal end. The inflatable member includes one or more grooves formed on its surface and one or more electrodes disposed in the grooves. Small holes in the grooves proximate each the one or more electrodes allow for the seepage of inflation fluid from the interior of the inflatable member. The one or more grooves may be longitudinal, circumferential, or helical. There may be one, two, or more electrodes in each of the one or more grooves. The grooves and the electrodes may be arranged to provide circumferential coverage. The grooves and the electrodes are arranged such that when the inflation member is inflated in a blood vessel, the electrodes are spaced from the wall of the blood vessel.

Some embodiments pertain to a system for nerve modulation including a device having a double-walled balloon on the distal end. The outer balloon may include one or more grooves as described above. One or more electrodes may be placed in the one or more grooves or under the one or more grooves between the outer balloon and the inner balloon. Small holes in the grooves proximate each the one or more electrodes allow for the seepage of inflation fluid from the interior of the outer balloon. The grooves and the electrodes are arranged such that when the inflation member is inflated in a blood vessel, the electrodes are spaced from the wall of the blood vessel. The outer balloon may be made from a more compliant material and the inner balloon may be made from a less compliant material.

The summary of some example embodiments is not intended to describe each disclosed embodiment or every implementation of the disclosure.

### BRIEF DESCRIPTION OF THE DRAWINGS

The present disclosure may be more completely understood in consideration of the following detailed description of various embodiments in connection with the accompanying drawings, in which:

FIG. 1 is a schematic view illustrating a renal nerve modulation system in situ.

FIG. 2 is an isometric view of the distal end portion of an exemplary renal nerve system.

FIG. 3 is a cross-sectional view illustrating the illustrating the distal end portion of an exemplary system in situ.

FIG. 4 is a detail cross-sectional view illustrating a portion of an exemplary renal nerve modulation system.

FIG. 5 is an isometric view of the distal end portion of an exemplary renal nerve modulation system.

FIG. 6 is an isometric view of the distal end portion of an exemplary renal nerve modulation system.

FIG. 7A is a schematic view illustrating the distal end portion of an exemplary renal nerve modulation system in situ.

FIG. 7b is a cross-sectional view of the system of FIG. 7A along the section indicated in FIG. 7A.

FIG. 7C is a schematic cross-sectional view of an exemplary renal nerve modulation system.

FIG. 8 is a schematic side view of an exemplary renal nerve modulation system.

FIG. 9 is a detail view of an exemplary renal nerve modulation system.

FIG. 10 is a schematic side view of an exemplary renal nerve modulation system.

FIG. 11 is a schematic cross-sectional view of an exemplary renal nerve modulation system.

FIG. 12 is a schematic side view of an exemplary renal nerve modulation system.

While embodiments of the present disclosure are amenable to various modifications and alternative forms, specifics thereof have been shown by way of example in the drawings and will be described in detail. It should be understood, however, that the intention is not to limit aspects of the disclosure to the particular embodiments described. One the contrary, the intention is to cover all modifications, equivalents, and alternatives falling within the spirit and scope of the present disclosure.

### DETAILED DESCRIPTION

For the following defined terms, these definitions shall be applied, unless a different definition is given in the claims or elsewhere in the specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimension ranges and/or values pertaining to various components, features, and/or specifications are disclosed, one of skill in the art, incited by the present disclosure, would understand desired dimensions, ranges and/or values many deviate from those expressly disclosed.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where ablation or modulation is desired such as nerve modulation and/or ablation near other vessel lumens. It is contemplated that the devices and methods may be used in other treatment locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pulmonary vein isolation, tumor ablation, benign prostatic hyperplasia therapy, nerve excitation or blocking or ablation, modulation of muscle activity, hyperthermia or other warming of tissues, etc. The disclosed methods and apparatus can be applied to any relevant medical procedure, involving both human and non-human subjects. The term modulation refers to ablation and other techniques that may alter the function of affected nerves and other tissue.

In some instances, it may be desirable to ablate perivascular renal nerves with targeted tissue heating. However, as energy passes from an electrode to the desired treatment region the energy may heat the fluid (e.g. blood) and tissue as it passes. As more energy is used, higher temperatures in the desired treatment region may be achieved, but may result in some negative side effects, such as, but not limited to, thermal injury to the vessel wall, blood damage, clotting, and/or electrode fouling. Positioning the electrode away from the vessel wall may provide some degree of passive cooling by allowing blood to flow past the electrode while still allowing the electrode elements to target nerves within about 2.5 mm of the luminal surface, where the perivascular renal nerves are located. An appropriate amount of energy may properly ablate the nerve tissue while causing little or no damage to the vessel wall or to deep tissue such as muscle tissue or the intestinal walls.

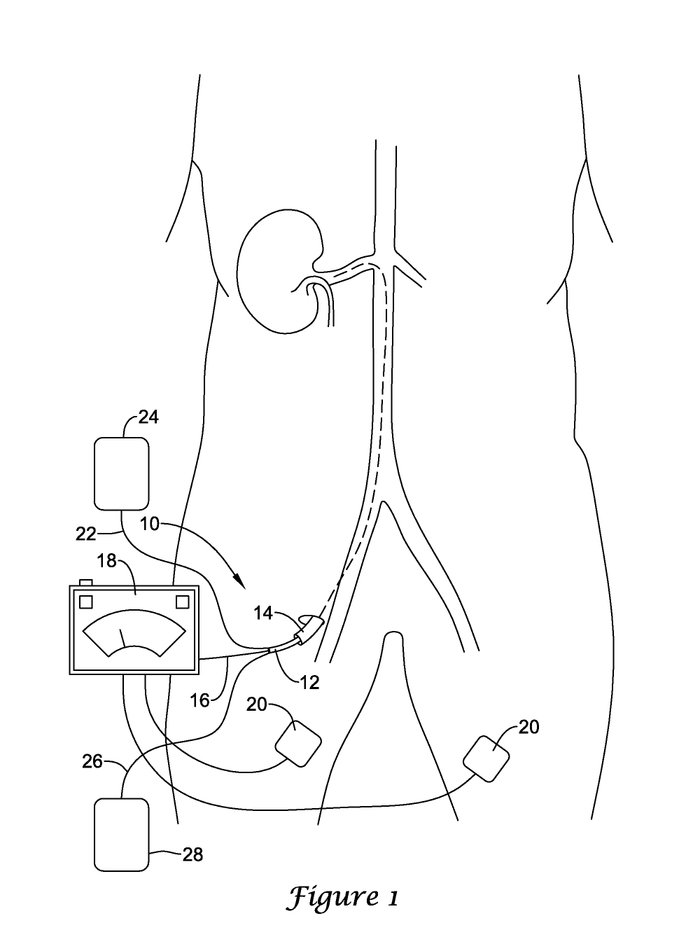

FIG. 1 illustrates a general overview of a renal nerve modulation system 10 as it may look during an operation. System 10 includes a renal nerve modulation device 12, which may be inserted percutaneously into a blood vessel through a guide catheter 14. A femoral approach is illustrated, though it will be readily appreciated that the devices and methods described herein may readily be used with other approaches such as a radial approach. The scope of the disclosure with regard to the distal end of device 12 will be better understood when the subsequent figures are discussed, below.

The proximal end of the device 12 is generally operatively connected via a control and power wire 16 to a control and power unit 18. The control and power wire 16 may include an appropriate number of separate conductors. For example, in some embodiments, each electrode or set of electrodes is operatively connected to the control and power unit 18 by separate conductors. Further, some embodiments may include one or more sensors, such as a thermocouple or pressure sensor, at the distal end region of the device 12. Each such sensor may be operatively connected to the control and power unit 18 by a separate conductor. All these conductors may be part of control and power wire 16.

The control and power unit 18 includes appropriate elements to control the device 12 and provide appropriate feedback. Such elements may include any standard electric and/or electronic system control elements. Examples of such elements include power supplies, switches, displays, programmable interfaces and the like. The control and power unit 18 may include monitoring elements to monitor parameters such as power, temperature, voltage, amperage, impedance, pulse size and/or shape and other suitable parameters as well as suitable controls for performing the desired procedure. In some instances, the power element 18 may control a radio frequency (RF) electrode. The electrode may be configured to operate at a frequency of approximately 460 kHz. It is contemplated that any desired frequency in the RF range may be used, such as, for example, from 400-900 kHz. However, it is contemplated that different types of energy outside the RF spectrum may be used as desired.

Device 12 may also be operatively attached by a fluid inlet lumen 22 to a fluid source 24 such as a syringe or a pump. The fluid from the fluid source is preferably a bio-compatible electrically conductive fluid such as an isotonic saline or the like. The fluid may also include a radiopaque agent. Some embodiments of the device may circulate the fluid through the distal end region of the device and thus may also include a fluid drainage lumen 26, which may, in turn, be attached to a fluid collection bag 28 or the like. Some embodiments, as discussed below, include more than one balloon in the distal end region, and thus the device may be operatively connected to more than one fluid source at the proximal end.

In some embodiments, the control and power unit 18 may further be connected to one or more return electrode patches 20, which, during operation, may be located on the abdomen or at another conventional location on the body. The proximal end of the system may also include other standard elements (not explicitly shown), such as a hub, a handle, a guide wire lumen, and the like. The system 10 may include other elements, not illustrated, such as guide wires, introducer sheaths and the like. The device 12 may be removably connected to any or all exterior elements.

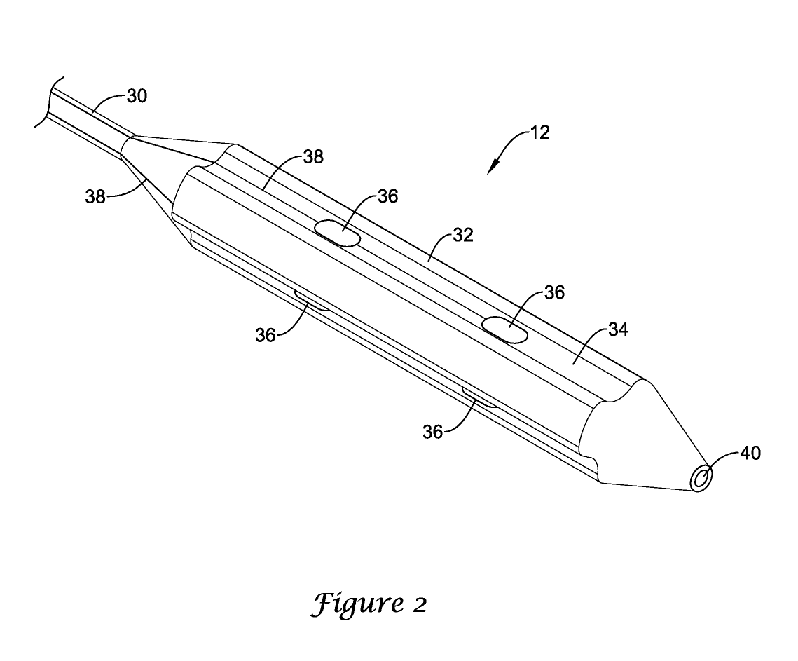

Turning now to FIG. 2, which illustrates the distal end region of an example device 12, according to a particular embodiment. The distal end region generally includes a balloon 32, which is at or near the distal end of a catheter 30 and connected thereto. The interior of the balloon 32 is fluidly connected to a fluid inlet lumen in the catheter 30. The catheter 30 may also include other lumens such as a guidewire lumen 40 and, if desired, a separate fluid egress lumen. The balloon 32 is movable, through the introduction and removal of an inflation fluid, between an inflated state (illustrated) and a collapsed state. When inflated, the balloon 32 may have a generally circular cross-sectional profile, interrupted by shallow longitudinal grooves 34. The particular embodiment illustrated includes three grooves 34, but may include fewer or more grooves 34 as desired. One or more electrodes 36 may be at the bottom of each groove. Each electrode 36 may be connected to the control and power unit by a separate conductor 38. Alternatively, sets of electrodes such as the sets formed by the electrodes in each groove may share a conductor 38. It can be appreciated that any combination of electrodes may share a power connection. For example, each of the two circumferential sets formed by the three distal electrodes and the three proximal electrodes may be powered as a unit such that one conductor 38 powers the proximal set and one conductor 38 powers the distal set. Alternatively, all electrodes may share the same power connection.

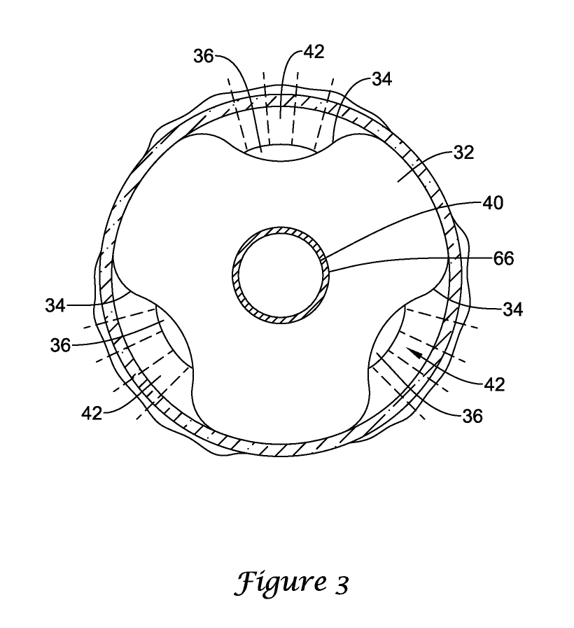

Variations on the size, shape, number and arrangement of the electrodes 36 are contemplated. For example, the embodiment illustrated shows the three proximal electrodes at the same longitudinal location and the three distal electrodes as the same longitudinal position. In other embodiments, the longitudinal position of the electrodes 36 may be staggered such that each electrode, or no more than two electrodes share the same longitudinal position. The shape of the electrode is illustrated as generally oblong, although any convenient shape may be used such as circular, oval, polygonal and the like. In some embodiments, it is preferable to avoid electrodes that have exposed sharp edges. The length (i.e. the dimension of the electrode along the longitudinal dimension of the device) to width ratio may vary as desired. For example, the ratio may be 1:1, 1.5:1, 2:1, 3:1, 5:1, 10:1 or other desired ratio. There may be more or fewer electrodes 36 per groove 34. For example, there may be one, two, three, four other desired number of electrodes 36 per groove 34. In some embodiments, one or more of the grooves 34 may be used solely for the purpose of maintaining blood flow and so there may be no electrodes in one or more grooves. The relationship between the electrode 36, the balloon 32 and the grooves 34 will be discussed with respect to subsequent figures such as FIG. 3, which is a distal end view of the device 12 of FIG. 2 in situ.

FIG. 3 illustrates device 12 as it might appear when inflated and ready to use for a therapeutic procedure. The device 12, when inflated, is preferably sized to center the device 12 within a blood vessel. The balloon 32 may be made from a compliant or semi-compliant material that may allow the balloon 32 to be used within blood vessels having a range of diameters. For example, a balloon having a nominal diameter of 6 mm may, through varying the inflation pressure, accommodate blood vessels having diameters of between 5 mm and 7 mm. Of course, devices of varying sizes to accommodate a range of blood vessels may be manufactured for use. In some embodiments, the device 12 may include a guidewire lumen 40 defined by a catheter wall 66.

When inflated for a procedure, the grooves 34 should provide a gap 42 between the electrodes 36 and the wall of the blood vessel. The gap may be any appropriate dimension. A gap of over 0.0010″ may be appropriate for some embodiments. In preferred embodiments, a minimum non-zero gap between the electrodes and the vessel wall is maintained. The broken lines in FIG. 3 generally indicate the transmission of RF energy from the electrodes through the wall of the blood vessel and into the nerve tissue.

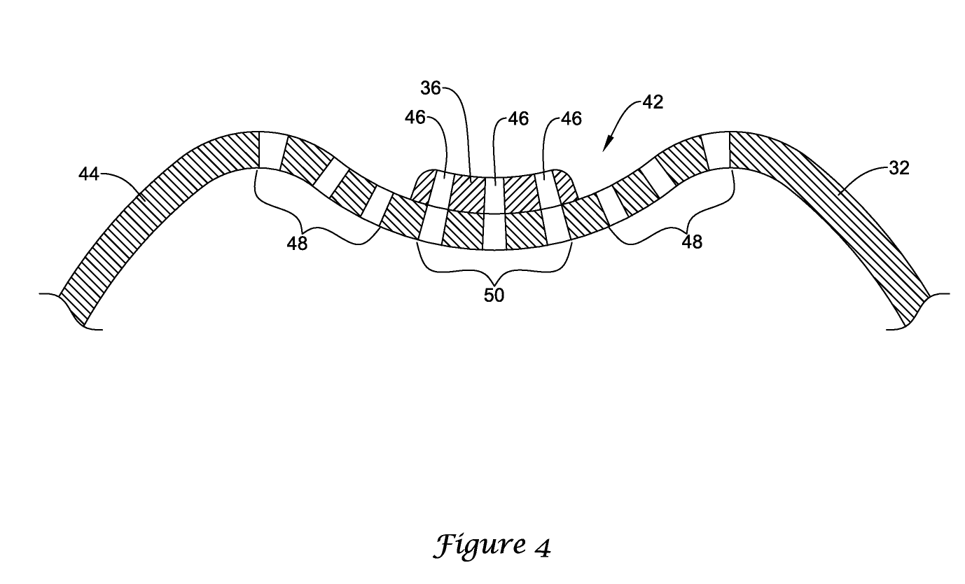

FIG. 4 is a detail view illustrating an electrode 36 and an associated portion of the wall 44 of the balloon 32 for a device 12 as illustrated in FIG. 2. The balloon wall 44 of a device 12 may include small holes 48, 50 proximate each operative electrode 36 of the device 12. Such holes may be in one or more of the side walls of a groove 34 as indicated at 48 or may be beneath an electrode 36 as indicated at 50, or both. The electrode 36 may also include holes 46 to permit egress of the fluid flow through holes 50. The size and quantity of such holes 46, 48, 50 should be such as to keep the total flow of fluid from the balloon cavity to a low figure. For example, holes sizes in the range of 10-20, 10-30, 20-30, or 20-40 microns may be appropriate. A total fluid flow of between 10-20 cubic centimeters (cc)/minute (min) or between 10-30 cc/min or under 50 cc/min may be appropriate in certain applications.

An example therapeutic procedure using the embodiment of FIG. 2 will now be described. A device 12 is inserted percutaneously into a renal artery using a femoral approach. The device 12 may be inserted using a guide catheter 14 and/or other standard devices and procedures such as a guide wire. The device 12 is inserted in its collapsed configuration through, for example, the guide catheter 14. The guide catheter 14 distal end may be positioned just proximal the desired location of the therapeutic procedure and the distal end region of the device may be advanced out of the guide catheter 14 and into position. Alternatively, the guide catheter distal end can be positioned at the desired location with the distal end region of the device 12 inside. The guide catheter 14 may then be withdrawn proximally, leaving the device 12 in place. Other standard delivery techniques may be employed as well. Once the distal end region of the device is in place, an inflation fluid, such as the saline discussed above, is introduced to inflate the balloon. In this embodiment, a high inflation pressure is not necessarily needed or desired. Accordingly, the balloon 32 may be inflated to a pressure of between 0-3 atmosphere (atm). When the balloon is inflated, the electrodes 36 are held by the shape of the grooves 34 away from the vessel wall, defining the gap 42 spacing the electrode 36 from the vessel wall. The inflation fluid will also begin to seep from holes 46, 48, 50 which may be present in the particular embodiment. At this point, the electrodes 36 may be activated to generate an RF current. The inflation fluid, vessel wall, and body tissue complete the circuit between the electrodes and any return electrodes. The RF current generates heat in the tissue to denervate the nerve tissue. At the same time, the inflation fluid seeping out from the balloon and the blood in the blood vessel flow past the vessel wall and transfer heat from the portion of the vessel wall nearest the lumen (i.e. the intima and proximate portions of the media) to prevent damage to the blood vessel wall. The RF energy may be provided at any effective level. For example, in some procedures, 1-15 watts for 1-2 minutes is effective. In some procedures, the device 12 is then withdrawn using conventional methods. In other procedures, the device 12 may be repositioned and the denervation procedure using the RF energy repeated. The reposition may aided by partially or completely evacuating the inflation fluid from the balloon cavity, to partially or completely collapse the balloon. The balloon may then be reinflated at the new location. In some renal nerve denervation procedures, it may be desirable to denervate a circumferential section of nervous tissue to interrupt the function of nerves running along the renal arteries. This may be a contiguous circumferential section or may include non-contiguous sections that are at different circumferential and longitudinal locations and are arranged such that any longitudinally extending nerve is interrupted by at least one denervated section. In some procedures, 80%, 90% or 95% coverage may be effective. In other words, in some procedures, denervating 80%, 90% or 95% of a contiguous or non-contiguous circumferential section may be adequate.

The above procedure was described as a unipolar procedure, where the electrodes 36 are used to radiate RF energy to one or more return electrodes 20 on the body of the patient. However, a device 12 is also suitable for use with bipolar procedures. In bipolar procedures, some of the electrodes 36 act as the return electrode(s), and in some bipolar procedures, the electrodes 36 that act as return electrodes alternate with the alternative current. Any device 12 where electrodes or sets of electrodes 36 are controlled and powered separately may be suitable for use in a bipolar procedure. In a bipolar procedure, the RF current passes from an electrode 36, through the fluid in the blood vessel, the wall of the blood vessel and the body tissue and then back to the control and power unit 18 through another electrode 36. Because unipolar and bipolar operation create different denervation patterns, it may be desirable to perform unipolar and bipolar operations during the same procedure.

Performance may be monitored during the procedure through the use of sensors in the distal end region and by monitoring the change in impedance of the current through the body. Appropriate sensors include temperature sensors such as thermocouples and pressure sensors. The control and power unit 18 may be programmed to dynamically respond to changes in the sensor measurements.

Further, while these example procedures have been described with respect to the embodiment of FIG. 2 described above, it can be readily appreciated that the procedure may be used with the other embodiments described in this specification.

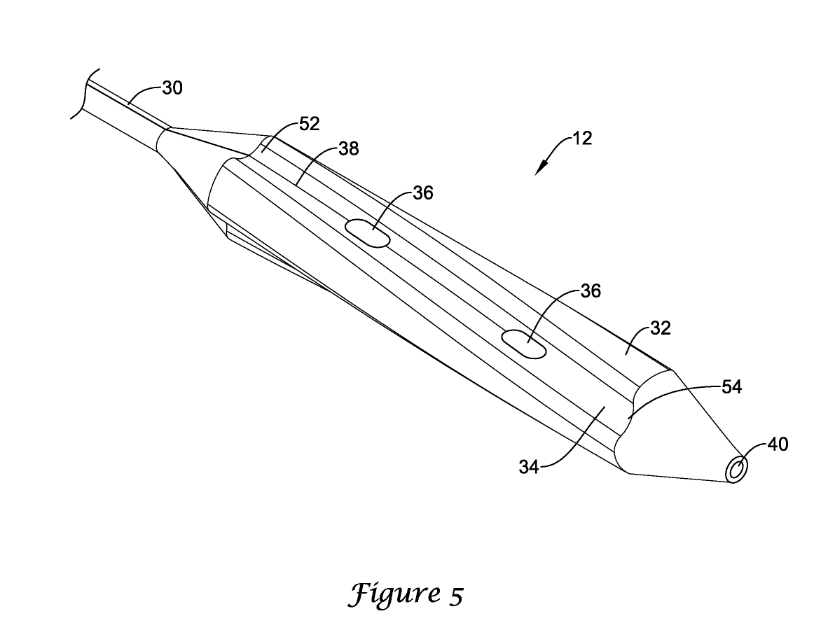

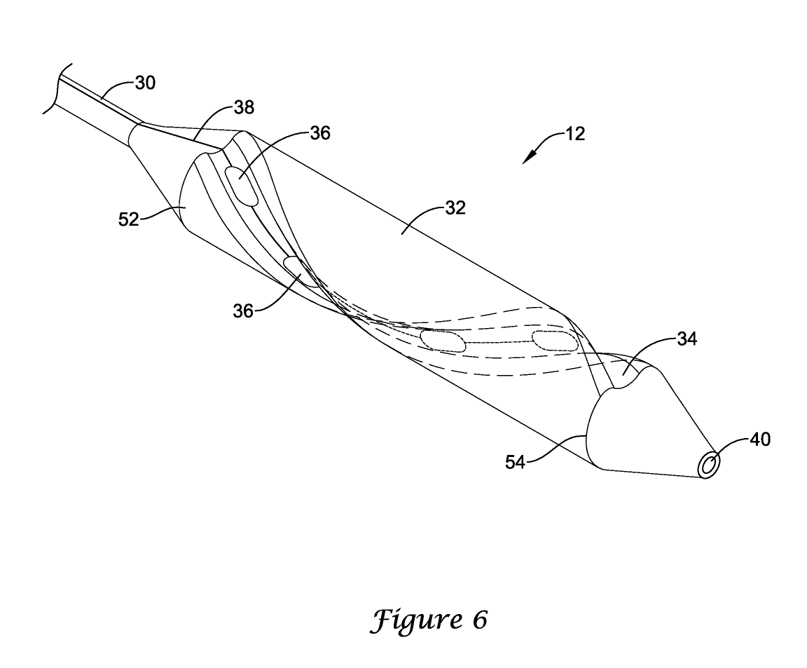

For example, FIGS. 5 and 6 are isometric views of distal end regions of devices 12 that are similar to that of FIG. 2, except as illustrated and described herein. The embodiment of FIG. 5 has three grooves 34 that extend along the surface of balloon 32 in a generally helical or spiral fashion. Electrodes 36 are disposed at the bottom of grooves 34. The helical arrange of the grooves allows for a wider circumferential distribution of the electrodes. For example, the six electrodes 36 illustrated in FIG. 5 may be arranged such that each is approximately 60° from its nearest two neighboring electrodes 36. In this fashion, the six electrodes 36 are equally distributed circumferentially around the body of the balloon 32.

FIG. 6 illustrates an embodiment having a single groove 34 that extends helically around the body of the balloon. In the particular embodiment illustrated, the groove 34 extends for a complete 360° loop. Of course, depending on the desired arrangement of the electrodes, the groove 34 may extend for less than a full loop or for more than a full loop, and may also be at any desired pitch or at a varying pitch. Four electrodes 36 are disposed in groove 34 and may be equally circumferentially spaced about the circumference of the balloon or have another desired arrangement.

Of course, variations as discussed above with respect to the previous figures are applicable to the embodiments of FIGS. 5 and 6 as well.

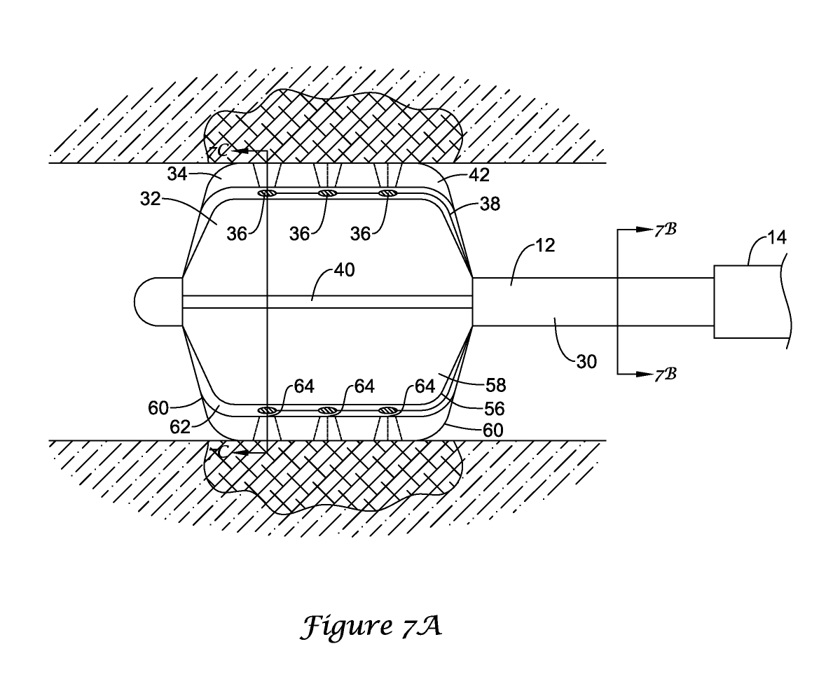

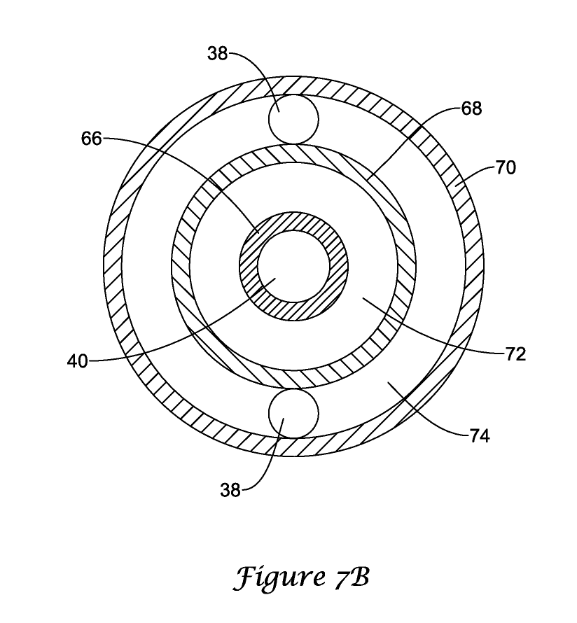

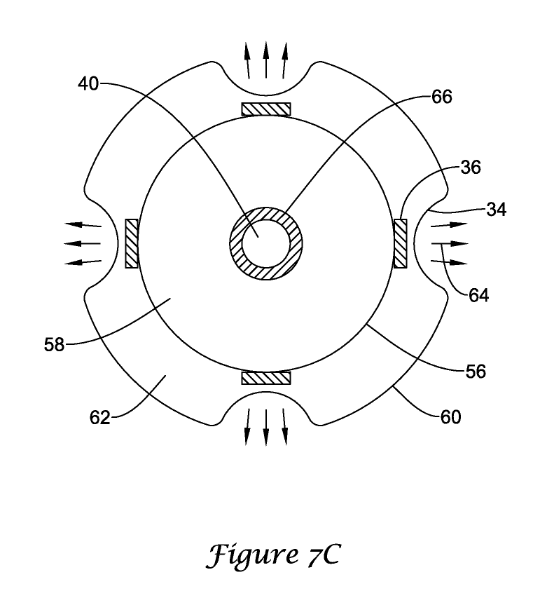

FIG. 7A is a schematic view of the distal end region of a device 12, with the device generally illustrated with a side view but having the balloon 32 illustrated in a schematic cross-sectional view. Device 12 is illustrated as being positioned in situ and extending from the distal end of a guide catheter 14. Device 12 includes a double-walled balloon 32 and has an inner balloon wall 56 defining an inner balloon cavity 58, and an outer balloon wall 60 defining an outer balloon cavity 62. The inner balloon may be made from a non-compliant or stiffer material. The inner balloon is preferably sealed (save for the connection to an inflation lumen) and has no openings to the outer balloon cavity or to the blood vessel. The inner balloon may have a generally cylindrical profile. The outer balloon may be made from a semi-compliant or compliant material and, as discussed below, preferably includes features such as grooves 34. As can be seen better with reference to FIG. 7C, which is a cross-sectional view of the balloon 32 taken at line 7C-7C in FIG. 4A, the outer balloon has four longitudinally extending grooves 34. The cross-sectional view of the balloon 32 in FIG. 7A shows the outer balloon wall 60 at the grooves 34 and also the portion of the outer balloon wall that is visible behind the grooves 34. The balloon 32 may include other desired features such as a guide wire lumen 40. Electrodes 36 and conductors 38 are generally disposed in the outer balloon cavity 62 and may be attached to the outer surface of the inner balloon wall 56 or fixed in another appropriate manner. Micro-pores to permit seepage of fluid from the outer balloon are disposed proximate the electrodes 36 and are generally indicated at 64. The grooves 34 define a gap 42 between the electrodes 36 and the outer wall when the balloon 32 is inflated.

FIG. 7B is a cross-sectional view of catheter 30, taken at line 7B-7B shown in FIG. 7A, illustrating a concentric arrangement of lumens 40, 72, 74 defined by catheter walls 66, 68, 70, which lumens are used as a guide wire lumen 40, inner balloon lumen 72 and outer balloon lumen 74. This arrangement is merely an example arrangement of lumens, and other arrangements are contemplated. For example, the lumens need not be concentric, or, in some embodiments, one or more return lumens might be desired. Further, other conductors 38 might be included for connection with additional electrodes or sensors.

This embodiment may used in the procedures and manner described above, except as noted herein. The nature of the double-balloon of this embodiment requires the inflation of both the inner balloon and the outer balloon. The inner balloon may be inflated to a high pressure, such as 3 atm, and the outer balloon may be inflated to a much lower pressure to softly and atraumatically center the distal end region in the blood vessel.

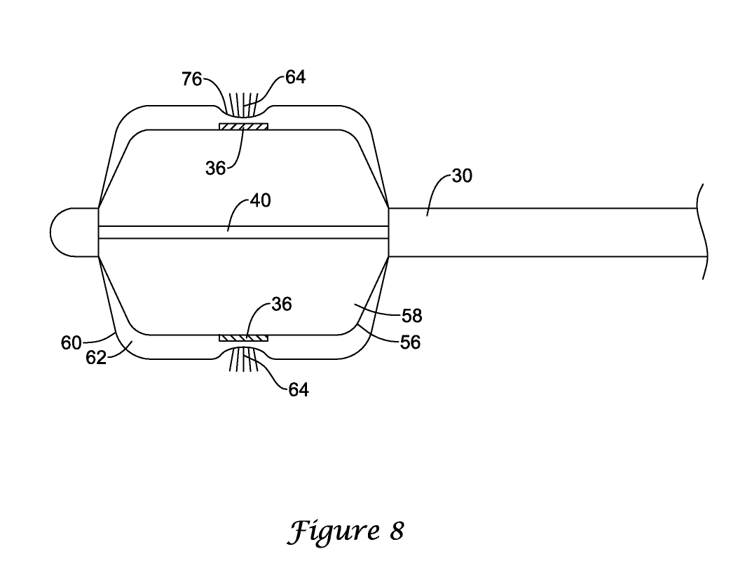

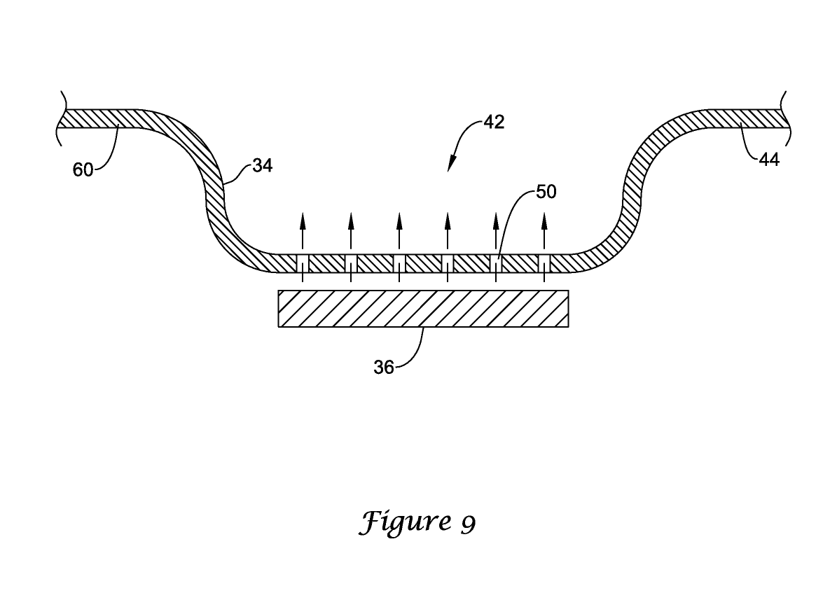

It can be appreciated that the variations discussed above, particularly with respect to the arrangement and number of grooves and of electrodes are applicable to the embodiment of FIGS. 7A-7C. For example, there may be more or fewer grooves, the grooves might extend helically, and/or the electrodes might be staggered longitudinally. FIG. 8 illustrates a variation of a double-walled balloon 32 embodiment where a plurality of electrodes 36 are arranged circumferentially on the outer surface of the inner balloon wall 56. The outer balloon wall has a circumferential groove 76 extending around the balloon over the electrodes 36. Reference numeral 64 indicates that micro-pores are disposed in the outer balloon wall 60 over the electrodes. FIG. 9 is a detail view of the circumferential groove of FIG. 8 and shows micro-pores 50 disposed in outer balloon wall 60 over the electrodes 36. Micro-pores 50 may be disposed elsewhere in the groove 76 proximate the electrode. For example, the micro-pores may be disposed in the side walls of the groove.

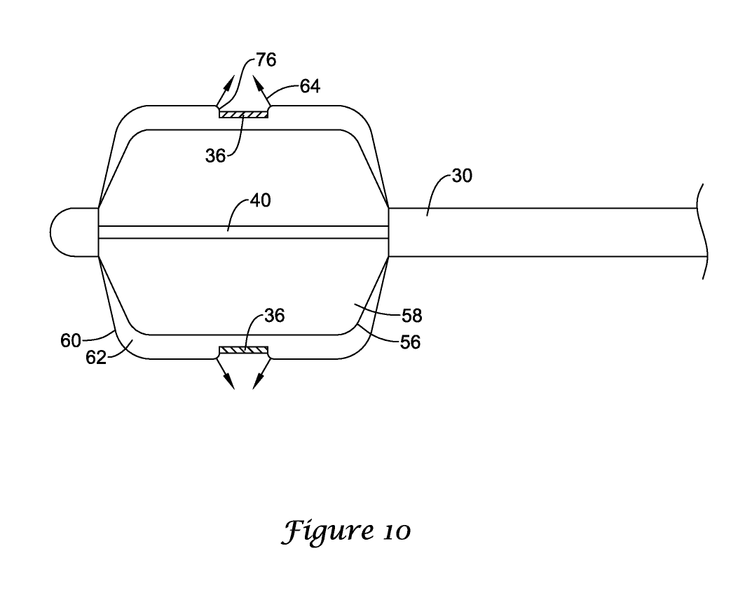

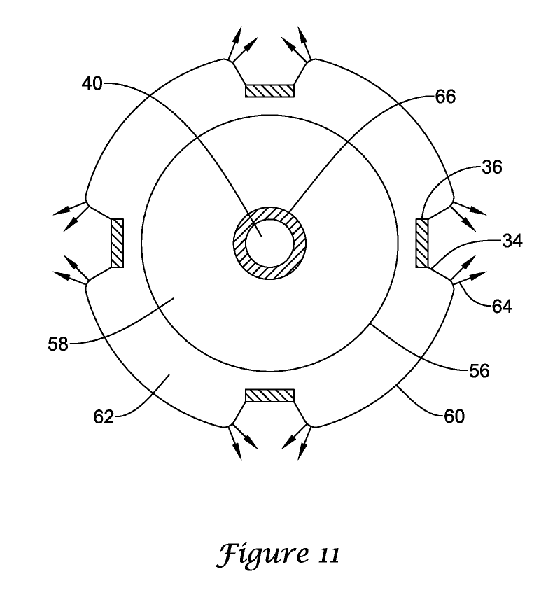

FIGS. 10 and 11 illustrate the distal end portion of double-walled balloon embodiments, where the electrodes 36 are on the outer surface of the outer balloon wall 60. FIG. 10 depicts an embodiment where the electrodes are arranged circumferentially about the balloon and the groove is also circumferential, as in FIG. 8, and FIG. 11 depicts an embodiment where the electrodes are arranged in longitudinally extending grooves, as in FIG. 7A. The outer balloon walls 60 are perforated with micro-pores indicated by the arrows and at reference numeral 64. It can be appreciated that the variations and methods of use discussed above are applicable to these embodiments as well.

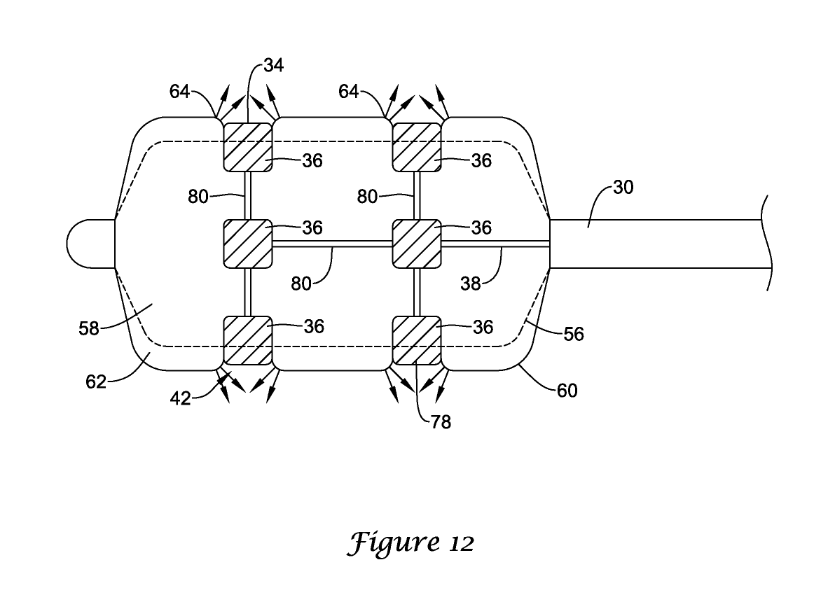

FIG. 12 illustrates the distal end portion of an embodiment that does not have grooves pre-formed into the balloon wall. Instead, the compliant outer balloon wall 60 is selectively restrained as it expands to bulge out around the electrodes 36 and thereby form grooves 34. Connectors 80 form the electrodes 36 into a network that restrains the expansion of portions of the balloon. For example, the circumferentially oriented connectors 80 that connect the two circumferential bands of electrodes form circumferential bands that limit the expansion of the balloon in those areas. The balloon, made from a compliant material, is free to expand in other locations and so expands around the electrodes and connectors to form the grooves 34. The outer balloon wall may preferably include micro-pores in the proximity of the electrodes, indicated at 64 and by the arrows, to allow fluid seepage from the outer balloon cavity 62. In some embodiments, the electrodes 36 and connectors 80 are formed as a single unit that expands when the balloon is inflated like an expandable stent. Thus, when the balloon is in the deflated configuration, connectors 80 may form a zigzag configuration to allow the balloon profile to diminish. In other embodiments, the connectors 80 may be replaced by circumferential bands of a non-compliant material of a preset diameter that are attached to the outer balloon wall 60 under or over the electrodes 36. The embodiment illustrated is a double-walled balloon embodiment like that of FIG. 7A, although it will be appreciated that the unique features of the FIG. 12 embodiment may be readily used with a single-walled balloon embodiment.

Balloons used in these embodiments having the preset grooves may be manufactured by blow molding. In some embodiments, the grooves are made less compliant, or stiffer, than the other portions of the balloon. This may be accomplished by making the wall of the balloon thicker in the areas of the grooves. For example, the wall thickness of the material of the grooves may be four times thicker than that of the other portions of the balloons. This may also be accomplished by adding a layer of higher durometer material to the groove areas.

Those skilled in the art will recognize that the present disclosure may be manifested in a variety of forms other than the specific embodiments described and contemplated herein. Accordingly, departure in form a and detail may be made without departing from the scope and spirit of the present disclosure as described in the appended claims.

What is claimed is:

1. A system for nerve modulation, comprising:
a catheter shaft having a proximal end, a distal end and lumen extending therebetween;
an inflatable member fluidly connected to the lumen of the catheter shaft proximate the distal end of the catheter shaft, the inflatable member having an outer wall defining a groove in an outer surface of the inflatable member; and
an electrode disposed in or under the groove.

2. The system of claim 1 wherein the groove extends longitudinally along at least a portion of the outer wall of the inflatable member.

3. The system of claim 1 wherein the groove extends in a helical manner along at least a portion of the outer wall of the inflatable member.

4. The system of claim 1 wherein the groove is a circumferential groove.

5. The system of claim 1 wherein the groove is one of a plurality of grooves.

6. The system of claim 5 wherein the plurality of grooves are regularly spaced on the inflatable member.

7. The system of claim 5 wherein the electrode is one of a plurality of electrodes and wherein each of the plurality of electrodes is disposed in or under one of the plurality of grooves.

8. The system of claim 7 wherein at least one electrode is disposed in or under each of the plurality of grooves.

9. The system of claim 1 further comprising a plurality of small holes in the outer wall of the inflatable member proximate each electrode.

10. The system of claim 9 wherein the plurality of small holes are in the groove.

11. The system of claim 9 wherein each of the plurality of small holes has a maximum width of between 10 and 20 microns.

12. The system of claim 1 wherein the electrode is in the groove.

13. The system of claim 1 wherein the inflatable member further comprises an inner balloon disposed within the inflatable member.

14. The system of claim 13 wherein the outer wall of the inflatable member comprises a compliant material and the inner balloon comprises a non-compliant material.

15. The system of claim 13 wherein the electrode is disposed between the outer wall of the inflatable member and the inner balloon.

16. A system for nerve modulation, comprising:
a catheter shaft having a proximal end, a distal end and lumen extending therebetween;
an inflatable member fluidly connected to the lumen of the catheter shaft proximate the distal end of the catheter shaft, the inflatable member having an outer wall defining a plurality of grooves in an outer surface of the inflatable member;
a plurality of electrodes disposed within the plurality of grooves; and
a plurality of holes in the outer wall of the inflatable member proximate each electrode.

17. A system for nerve modulation, comprising:
a catheter shaft having a proximal end, a distal end and at least one lumen extending therebetween;
an outer balloon fluidly connected to the catheter shaft proximate the distal end of the catheter shaft, the outer balloon having an outer wall defining a groove in an outer surface of the outer balloon;
an inner balloon fluidly connected to the catheter shaft proximate the distal end of the catheter haft and disposed within the outer balloon; and
at least one electrode disposed in or under the groove.

18. The system of claim 17 wherein the at least one electrode is disposed on an outer surface of the inner balloon and disposed under the groove.

19. The system of claim 17 further comprising a plurality of holes in the outer wall of the outer balloon proximate the at least one electrode.

20. The system of claim 17 wherein the groove is one of a plurality of grooves.
